(19) United States
(12) Patent Application Publication
(10) Pub. No.: US 2007/0265518 A1
(43) Pub. Date: Nov. 15, 2007

(54) Method and arrangement for locating a medical instrument at least partly inserted into an object under examination

(76) Inventors: Jan Boese, Eckental (DE); Norbert Rahn, Forchheim (DE); Bernhard Sandkamp, Eriangen (DE)

Correspondence Address: SIEMENS CORPORATION;INTELLECTUAL PROPERTY DEPARTMENT, 170 WOOD AVENUE SOUTH, ISELIN, NJ, 08830, US

(21) Appl. No.: 11/522,262

(22) Filed: Sep. 15, 2006

(30) Foreign Application Priority Data
Sep. 16, 2005 (DE) 10 2005 044 338.9

(51) Int. Cl. A61B 5/05

(52) U.S. Cl. 600/407

(57) ABSTRACT

The invention relates to a method for locating a medical instrument (2a, 2b) at least partly inserted into an object under examination (U), there being acquired (11, 13) images of the object under examination (U) which capture the medical instrument (2a, 2b). A method which requires as little user interaction as possible can be provided by virtue of the fact that an image is acquired under first acquisition conditions and a substantially identical image is acquired under second acquisition conditions (11, 13), and the fact that the medical instrument (2a, 2b) is located from a subtraction (14) of image data sets of first and second acquisition conditions.

The invention relates to a method for locating a medical instrument at least partly inserted into an object under examination in accordance with the preamble of claim 1, and also to an arrangement for carrying out the method.

In the course of a medical intervention in an object under examination, such as a human or animal body, the condition of the object under examination is monitored at the relevant medical workstation commensurately with the risk involved in the intervention. As a rule, bodily functions of the object under examination are recorded for this purpose. If an intervention takes place with a medical device at least partly inserted into the object under examination, not only the patient's condition but also the position and/or location of the medical instrument inside the object under examination are of interest.

Medical instruments include, for example, catheters, guide wires, stents, venous locks, biopsy needles, endorobots or other means at least partly inserted into the object under examination in the course of medical interventions. In order to enable the site and/or three-dimensional orientation of the medical instrument to be established, a method is needed in conjunction with a device arrangement which enables the medical instrument to be located, that is to say, the position and/or, as appropriate, location to be determined. The medical staff also need to be able to determine the positional variations and/or locational variations of the medical instrument on an ongoing basis.

A method of this kind can increase the precision of the intervention, reduce injury to the object under examination and improve patient safety. The site and location of a medical instrument are typically determined by means of images of the object under examination which capture the instrument.

The professional journal article entitled “Ein Modellbasierter Ansatz zur Lokalisation von Basket-Kathetern für endokardiales Mapping” [“A model based approach for localization of basket catheters for endocardial mapping”], published in Biomedizinische Technik, volume 45, supplementary volume 1, 2000, describes a method provided for the three-dimensional location of a basket catheter and electrodes attached thereto in a volume data set, for example a data set of the human thorax. To locate the basket catheter in the volume data set, images are acquired, for example by means of a calibrated magnetic resonance method. Image structures which have been acquired and which are generated inter alia by a basket catheter are recognized by means of filters which extract the first derivative from images. The second derivative (Hesse matrix) of the image data provides information on the topology of the two-dimensional image.

However, determination of the position of the basket catheter electrodes requires user interaction involving manual marking of the catheter strings. A further disadvantage of this method is the fact that further evaluation of the image data requires a three-dimensional computer model of the basket catheter so that the position of the basket catheter electrodes can be visualized.

The unexamined German application DE 100 04 764 A1 also discloses a method for determining the position of a medical instrument. The method consists of a pre-operative phase which generates a three-dimensional image set prior to the medical intervention, and of an intra-operative phase in which two-dimensional images are acquired using an X-ray device during an intervention. A position-measuring device is also provided for the extracorporeal determination of the position of the medical instrument. Data correlation of the pre-operatively and intra-operatively generated image data is also used to calculate the position of the medical instrument within the three-dimensional image data set.

One disadvantage of this method is that it is possible to locate only instruments which either have externally visible markings or are provided with special, e.g. electromagnetic, position sensors. In particular, instruments such as catheters and guide wires which adapt their location to internal structures of an object under examination cannot be located by means of the disclosed optical position-measuring device.

The object of the invention is to provide a method of the type described in the introduction which requires as little user interaction as possible.

This object is achieved with the location method of the type described in the introduction, in that an image is acquired under first acquisition conditions and a substantially identical image is acquired under second acquisition conditions, and in that the medical instrument is located from a subtraction of image data sets of first and second acquisition conditions. This method enables the position and/or location of a medical instrument to be automatically recognized, and thus reduces the burden on the medical staff.

The medical instrument is acquired with the aid of an imaging examination device under first acquisition conditions and under second acquisition conditions. The first acquisition conditions and second acquisition conditions differ by virtue of a modified imaging of the instrument, that is to say a modification in the acquisition conditions e.g. on, in or around the instrument; they are otherwise substantially identical, e.g. size of the image segment, examination device acquisition position, examination device imaging parameters, etc. Image subtraction is used to eliminate background information of the image—usually information concerning the object under examination—which interferes with the location of the medical instrument. The instrument can thus be identified and located.

The image acquired consists of a sum of pixels which can be clearly detected by means of their coordinates. To each pixel there is assigned an associated intensity representing the result of the data acquired concerning the object examined. The images with and without modified acquisition conditions or instrument situation are thus intended to be substantially identical in content and acquisition conditions where the background is concerned, that is to say, as far as possible the acquisition conditions for the images are intended to differ only in the generated variation on, in or around the instrument.

In an advantageous variant of the invention, the image is acquired under one of the two acquisition conditions without contrast medium supplied, and the substantially identical image is acquired under the other acquisition conditions with contrast medium supplied. The image is thus acquired under the first acquisition conditions without contrast medium supplied and the substantially identical image is acquired under the second acquisition conditions with contrast medium supplied. This can, however, also be reversed. The instrument clearly stands out from the background as a positive or negative image by virtue of the enhancement of the contrast, and it is isolated and subsequently located by means of image subtraction.

The term “contrast medium” here is understood to mean all media that permit the contrast to be modified in an image for a given examination device, the imaging conditions of the examination device remaining unchanged. The supply of contrast medium can be automated or manually initiated, e.g. with an injection system, with allowance for system parameters that can be selected and adjusted by the user. For image acquisition it is advantageous for the image acquisition system to be linked to the injection system.

Image acquisition processes are always initiated if, as a result of the supply of contrast medium or supply of rinsing medium, the instrument contrast is maximal or minimal relative to the surrounding area, according to how the contrast medium supply or rinsing medium supply is regulated. Contrast media have been used in medical technology for many years. It is therefore relatively easy to use contrast media to modify the instrument situation in practice. Furthermore, as a result of this long experience with contrast media, such a procedure involves little risk to the patient.

In a further preferred variant of the invention, the contrast medium is supplied in such a way as to produce a contrast enhancement in the area surrounding the outside of the medical instrument. The contrast medium is supplied by means of, for example, an injection into the vascular system of the object under examination. As a result of the distribution of the contrast medium in the object under examination the vessels around the medical instrument also contain the contrast medium supplied and, in the image acquired, enhance the contrast between the area surrounding the medical instrument and the medical instrument itself. The instrument can thus be clearly identified by image subtraction of the images acquired. The contrast medium concentration selected should usefully be such that the object under examination suffers as little exposure as possible but should be adequate for instrument recognition purposes.

In an alternative embodiment of the invention, the contrast medium is supplied in such a way as to produce a contrast enhancement inside the medical instrument.

The object under examination is thus not directly exposed to the contrast medium, so the choice of contrast medium and/or the concentration of the contrast medium can be adapted to the desired contrast enhancement. Mercury can thus, for example, conceivably be used as a contrast medium for X-ray examinations.

For the purpose of receiving and guiding the contrast medium or rinsing medium in the medical instrument, the medical instrument is provided with a lumen which is connected to a contrast medium dispenser device by means of a line system. Contrast medium can thus be safely supplied to and removed from the instrument. A rinsing solution can be supplied to purify the lumen in the medical instrument if contrast medium need not be used, or is not to be used, at least temporarily in the further procedure. The contrast medium in the instrument and the resulting contrast enhancement relative to the outside surrounding area during image acquisition enables location to take place by means of image subtraction. It is also possible to use a combination of contrast media, that is to say X-ray positive and X-ray negative contrast medium, for image contrast enhancement, for example by supplying the object under examination and the medical instrument with oppositely acting contrast medium.

In a further preferred embodiment of the invention, the first acquisition conditions differ from the second acquisition conditions by virtue of the position and location of an instrument marking visible on the image. A radioopaque marking or sequence of markings can thus be integrated on or attached to, for example, the instrument tip, for example in the form of a small metal plate. For location purposes there are acquired some two substantially identical images which differ slightly, for example in the position of the medical instrument provided with radioopaque markings. Subtraction of the images produces segments of the instrument which are in a radioopaque form and the position and/or location of which is displaced relative to the other image. The image structures formed can contain just a few pixels but nevertheless ones which identify the medical instrument. The positions of the markings on the instrument are known from the outset. The position, for example of the tip of the instrument, can therefore be identified from the image structures produced.

In particular, there can also be provided collapsible and/or rotatable instrument tips which likewise permit the acquisition of two substantially identical images by modifying the instrument situation. The images then differ, for example, by virtue of the rotation and/or folding over of a marker on the instrument tip. After subtraction, structures which identify the tip of the instrument are thus produced in the resulting image. There can thus be attached to the instrument tip, for example, a small metal plate, the surface normal of which is substantially parallel to the propagation direction, e.g. of the X-rays, in the first acquisition process; it is then rotated and, in the second acquisition process, is perpendicular to the propagation direction of the X-rays. This is especially easy to achieve by rotation of the instrument about its longitudinal axis.

The marker location and/or marker position can be modified, for example mechanically or electrically. In the case of automated instrument control systems, the instrument situation can, in particular, be modified by an alignable instrument tip. Two data sets can thus be produced with a different alignment of the instrument tip. Here again the position and/or location of the instrument can be clearly determined following subtraction. An advantage of the use of instrument markers and the like is the fact that no injection systems are needed, so the method can be carried out more easily, more compactly, without the use of contrast medium, and more cheaply.

In a further advantageous embodiment of the invention, the first acquisition conditions and/or second acquisition conditions are initiated in a time-adjustable manner. During the intervention the contrast medium, for example, can be only temporarily present in the medical instrument and/or the object under examination, for example in specifiable periodic or aperiodic time intervals for a specifiable duration.

A stationary contrast medium flow or a static contrast medium can likewise be provided, for example, in the instrument. The timing of the contrast medium supply can be specified and modified by the medical staff as the situation dictates. The contrast medium and rinsing medium can, for example, be supplied periodically where contrast medium is used in the area surrounding the medical instrument.

After a maximum contrast medium concentration occurs around the instrument, the concentration decreases over time. The provision of a variation in the acquisition condition by means of rinsing agent is therefore dependent on the image acquisition frequency. Whether or not rinsing is necessary or is to be carried out to vary the acquisition conditions depends on the desired image frequency for location of the instrument. Similarly the instrument markings, for example, can be modified periodically or aperiodically. The modifications in the first acquisition conditions and second acquisition conditions can each be adapted to the particular intervention. The control of acquisition conditions can be automatic or user-controlled.

In a preferred embodiment of the invention, the images are acquired as a two-dimensional projection of the object under examination, and a two-dimensional position and/or location of the medical instrument is determined therefrom. For this purpose it is possible to use known means for carrying out a two-dimensional projection, e.g. X-ray methods. The projection of the object under examination, for example provided by X-ray apparatus, thus supplies for each pixel an integral intensity value which is determined from the local linear attenuation co-efficients of the object under examination in the projected direction through the object under examination. As a result of the use of known apparatus for carrying out the new method, few if any additional costs are likely to be incurred by the use of the method.

In a further advantageous embodiment of the invention, a three-dimensional position and location of the medical instrument is identified from at least two different image projections of the examination arrangement. The two different image projections each capture the medical instrument as a two-dimensional projection and differ from each other at least in the direction of projection through the object under examination. For the identification of the three-dimensional position and/or location of the medical instrument there is provided an arithmetic logic unit which provides the required information, that is to say the three-dimensionally represented site and/or location of the medical instrument in the object under examination, from the data of the different two-dimensional projections, for example by means of rear projection. With the aid of this device in conjunction with the applied method the medical staff can, in real time or at a display time close in time to the time of measurement, observe the three-dimensional site and/or location of the medical instrument, the instrument and the object under examination being imaged simultaneously.

In an alternative embodiment of the invention, the images capturing the medical instrument are acquired as three-dimensional data of the object under examination, and a three-dimensional position and/or location of the medical instrument is determined therefrom. The three-dimensional volume data set can be identified, for example, by means of a magnetic resonance device. For the location of the medical instrument according to the invention there is provided an arithmetic logic unit which identifies the required information, that is to say the three-dimensionally illustrated site and/or location of the medical instrument in the object under examination, from the data in the volume data set. The rear projection of two-dimensional data thus becomes unnecessary.

In a further preferred embodiment of the invention, a plurality of views are identified for a three-dimensional position and/or location of the medical instrument. It is possible for, for example, freely rotatable three-dimensional representations of the arrangement of the medical instrument in the object under examination to be generated without further image acquisition, for example using software. This gives the medical staff an optimal view of the above-mentioned arrangement and can reduce stress on the object under examination since fewer examination steps are required. Furthermore, the use of software facilitates contrast enhancement or the editing of further image characteristics, such as an image segment enlargement of a relevant area of an image.

In a further preferred embodiment of the invention, a starting position and/or starting location of the medical instrument is determined by image subtraction, and positional variations and/or locational variations of the medical instrument are captured by at least one preset number of image acquisition processes without image subtraction. Image recognition following determination of the starting position and/or starting location enables the positional variation and/or locational variation to be determined without carrying out image subtraction. For this purpose it is possible to use, for example, filter methods or methods which, following determination of the starting position and/or location, enable the site and/or orientation of the medical instrument to be recognized or determined without further image subtraction, especially including methods which are not image-based. Determination of the position and/or location without a further supply of contrast medium enables a saving to be made on contrast medium and the execution of the method to be generally speeded up by a reduction in the number of requisite method steps and also in the amount of image processing. If, as a result of background information and/or a rapid, possibly undefined movement of the medical instrument, the position and/or location display of the medical instrument is lost, it can be useful to re-initialize the location of the medical instrument according to the invention by means of a modified instrument situation. Generally speaking, at any desired time that the medical instrument is located at least partly inside the object under examination, it is possible to initialize location or positional variations and/or locational variations.

In a further advantageous variant of the invention, the images are acquired using a magnetic resonance method. A magnetic resonance method used with, for example, a nuclear spin tomograph is suitable for acquiring the necessary images for the location method. Alternatively, the images can be acquired using an X-ray method. A possible device for carrying out an X-ray method is, for example, an angiography device which generates the images for the location method. It is also possible for the images to be acquired using an ultrasound method. An ultrasound method, for example applied in a sectional view method, also referred to as B scan ultrasonography, is likewise suitable for acquiring the necessary images for the location method. Contrast media that can be used in this context should usefully be selected according to the image acquisition device used in conjunction with the location method.

In a further advantageous embodiment of the invention, at least one visible patient marking is captured on the acquired images. The patient marking is used as a reference point or control point for the image acquisition processes carried out. A recognizable reference point on substantially identical images which differ by virtue of a modified instrument situation enables matching image areas of the two images to be subtracted from each other with correct positioning. This is especially of interest whenever complete matching of the image area of the two images cannot be achieved.

In a further advantageous embodiment of the invention, the starting position and/or starting location and the positional variation and/or locational variation of a plurality of medical instruments are determined sequentially. This enables a particular instrument to be located when a plurality of instruments are inserted at least partly into the object under examination.

To this end, the medical instruments are controlled consecutively, that is to say sequentially, first with contrast medium and then with rinsing medium, for example by means of an electronic controller. The particular image is acquired while the contrast medium is in the medical device. Only the medical instrument controlled with contrast medium is therefore clearly visible, that is to say with contrast enhancement, on the acquired image. The positional data and/or the location of the instrument are saved by a memory device, and the instrument is cleaned of contrast medium using a rinsing medium. The next medical instrument is then captured and located by means of image acquisition processes, the data stored and the contrasted instrument rinsed.

This procedure is repeated until all the required medical instruments are initialized. Equally, instead of or together with acquisition processes with a different contrast medium situation, each instrument can also be initialized with modifiable instrument markings, instrument alignments, instrument positions or instrument locations, etc. The method for locating the medical instruments can then be used for each medical instrument. A plurality of at least partly inserted medical instruments can therefore be tracked independently in parallel in an object under examination and can be displayed in their three-dimensional location in the object under examination.

In a further advantageous embodiment of the invention, the images are acquired with the object under examination or its parts in the same condition of motion. When an examination is carried out on organs which, by virtue of their function, do not keep still, for example the heart or a lung, or even whenever the whole object under examination is in motion, the images acquired during the examination may not be substantially identical.

For example, where the image acquired is of the instrument in the heart and of the patient outwardly in a position of rest, at one time a systolic state of the heart may be captured and at another time a diastolic state of heart, thus making image subtraction more difficult. To lessen this problem, image acquisition processes can take place whenever the object under examination is in the same condition of motion, for example whenever the patient's exhalation phase has been completed. Thus images which are as identical as possible can be acquired, and the accuracy of the information contained in the image, that is to say the image content, can be increased.

To enable images to be acquired whenever the object under examination is in the same condition of motion, there is provided a controller which, on the basis of the data relating to the patient's state, controls image acquisition and the supply of contrast medium, that is to say, triggering of the image acquisition processes. Nevertheless, movements of the object under examination or its parts can be reflected in the acquired image as movement artefacts, that is to say image displacements or image unsharpnesses in image sub-areas as a result of movement, for example whenever a considerable change in the patient's state occurs during the examination period, for example in the exposure time to X-ray apparatus. This can likewise hinder the location of the medical instrument.

In a further advantageous embodiment of the invention, background deviations of substantially identical images are corrected by means of image-based elastic registration. This may be necessary in relation to the removal of movement artefacts, despite appropriate precautions. Known algorithms such as image-based elastic registration can be used for this purpose. This improves the informational content of the images acquired and thus affects the accuracy of the location method.

A combination of contrast medium supply and the use of at least one instrument marking for locating the instrument can be an advantageous means of improving the quality of location.

Further advantages of the method according to the invention in conjunction with an arrangement suitable for carrying out the method will emerge from an exemplary embodiment explained in more detail below with reference to the drawings. These provide diagrammatic illustrations as follows:

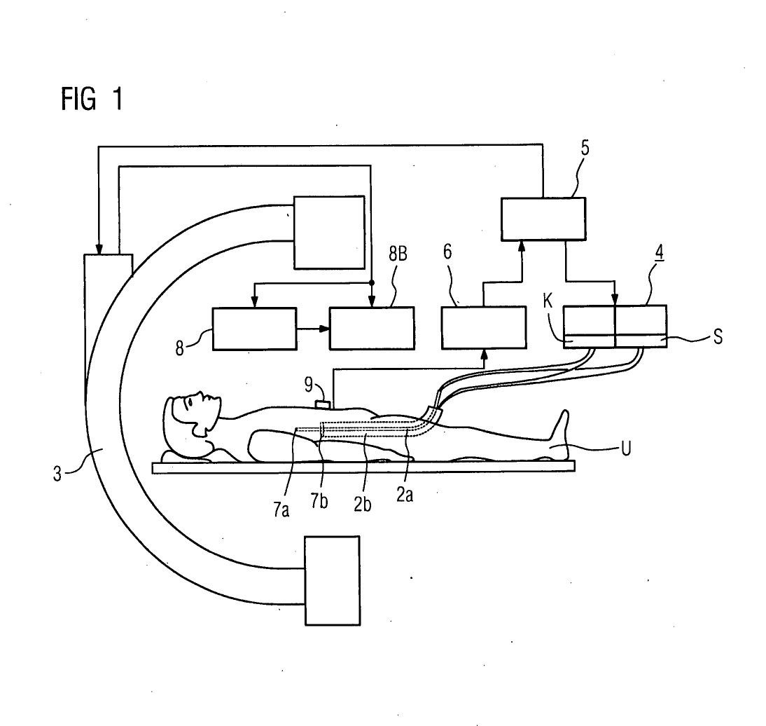

FIG. 1 an arrangement for carrying out the method according to the invention,

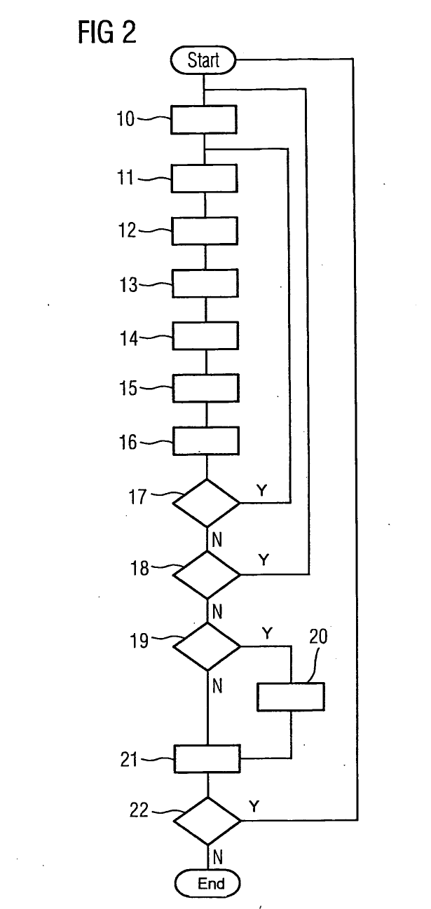

FIG. 2 a flow diagram showing the operations involved in the method according to the invention.

FIG. 1 shows an arrangement in the form of a medical workstation at which a medical intervention can be carried out on an object under examination U. Here two medical instruments, for example in the form of a balloon catheter 2a and a guide catheter 2b, are partly inserted into the object under examination U. The catheters 2a and 2b are located using an imaging examination device 3, which is here shown in the form of a monoplane C-arm X-ray system. The images acquired using the examination device 3 are visually displayed on an image system 8B. The instruments 2a and 2b each have a lumen through which a liquid can flow and which is tightly shut off from the surrounding area.

The lumen can guide a contrast medium K, or a rinsing medium S which is provided to rinse the contrast medium K out of the lumen. The lumen of the medical instruments 2a and 2b is connected via an incoming line and an outgoing line to a device for supplying and removing contrast medium K and rinsing medium S, here in the form of an injection system 4. The injection system 4 also incorporates an injector with contrast medium K and rinsing medium S, which can be supplied as necessary to each medical instrument 2a or 2b selectively or to both simultaneously. In addition to the examination device 3 and the injection system 4 there are provided means 6 for monitoring the patient's state and a data processing system 8.

The examination device 3, the injection system 4 and also the monitoring means 6 are each connected to a controller 5. The monitoring means 6 permit measurement of blood pressure, heart rate, respiration rate, blood gas values, etc. In co-operation with the controller 5 the measured values are used as a criterion for selecting the time when image acquisition will take place.

The data processing system 8 is used to evaluate the images acquired with and without contrast medium K but otherwise substantially identical; the said system is thus used inter alia for image subtraction, 2D location and 3D location of the medical instruments 2a and 2b. Radioopaque metal markings 7a and 7b on the instruments 2a and 2b respectively are also used for this purpose. A patient marking 9 is provided as a control point for image subtraction to allow for a modified patient position and/or patient location.

The data that are to be processed in the data processing system 8 are communicated to the data processing system 8 by the examination device 3. In conjunction with the data processing system 8, a memory unit for data save and further data sources, for example electrophysiological recording systems, 3D workstations, etc., can be present to enable different data to be combined with the position and location of the medical instruments 2a and 2b. The result of the data processing is displayed for the medical staff by means of the image system 8B.

The method steps shown in FIG. 2 will be described below in conjunction with the arrangement shown in FIG. 1; reference characters of arrangement components relate to FIG. 1. For the location of a medical instrument 2a or 2b in an object under examination U, a first method step 10 initially sets the projection direction in which the images are to be acquired by the examination device 3. An image capturing the medical instrument 2a is then acquired in a method step 11 under first acquisition conditions. At the same time, the patient's state at the time of image acquisition can be recorded by the monitoring means 6 through the controller 5. The image content comprises background information generated by the physiological structure of the object under examination U and also the medical instrument 2a or 2b, which is barely or not visible as a result of this background information. In a further step 12 contrast medium K is supplied to the instrument 2a by means of the injection system 4, which produces a variation in the first acquisition condition.

In the next method step 13 a further image of the instrument 2a in the object under examination U is acquired under second acquisition conditions. The information provided by the monitoring means 6 can be used to generate a substantially identical image to that generated in method step 11. The medical instrument 2a or 2b stands out from the background information and is recognizable as a result of the contrast medium K supplied. It is generally possible to interchange the sequence of image acquisition processes in relation to the contrast medium conditions.

Image subtraction of the images acquired in method steps 11 and 13 is then carried out in method step 14. The interfering background information is thereby greatly reduced or completely removed. The medical instrument 2a or 2b is now visible in the image as a remaining structure and can be captured, for example with an automatic recognition means. The image subtraction takes place in the data processing system 8. These data enable two-dimensional location of the medical instrument to be carried out in the initially set projection plane. The result can be displayed on the image system 8B in a method step 21.

The method can equally be used to carry out two-dimensional location of the medical instrument 2a in two or more planes. For this purpose, the method step 14 is followed by a decision step 18 in which the acquisition of the medical instrument 2a in the object under examination U from at least one further projection direction can be selected. The new projection direction is set in a method step 10. The method steps 11 to 14 then take place as described above in the newly selected projection direction.

If, in method step 18, the decision is taken not to modify the projection direction further, the two-dimensional representation of the medical instrument 2a can be displayed for the number of selected projection directions in method step 21.

In addition, in a method step 20, for the two-dimensional case there can be provided data preparation which, for example, evens out image displacements using image-based elastic registration. Alternatively, image projections of two different projection directions can be acquired simultaneously by using a biplane C-arm X-ray system.

Three-dimensional location of the medical instrument 2a with the present arrangement of a C-arm X-ray system 3 requires image data sets from at least two different projection directions unless three-dimensional data are obtained directly. If these data are available for a medical instrument 2a as per decision step 18 and after repeating method steps 10 to 14, a three-dimensional display can be selected in a decision step 19. To produce the display the image data sets are processed by means of rear projection in a method step 20 and displayed three-dimensionally on the image system 8B in a next method step 21.

If a plurality of medical instruments 2a and 2b are to be located in an object under examination U, acquisition of the first instrument 2a in a first projection direction as per method steps 10 to 14 is followed, in a further method step 15, by a data save. This can be done, for example, by filing the data in a memory means.

In a method step 16 the instrument 2a, to which contrast medium K was supplied in method step 12, is then purified from contrast medium K by means of a rinsing medium S. If, according to decision step 17, further instruments 2b are to be located, the method steps 11 to 16 are repeated for the further instruments 2b.

If all the instruments 2a and 2b are located in one projection direction, according to decision step 18 all the instruments 2a and 2b can be captured in a further projection direction. Alternatively, for each individual instrument 2a or 2b different projection directions can first be captured and then the next instrument 2a or 2b can be captured over a plurality of projection directions.

According to the generated data, the display for the location of the medical instruments 2a and 2b in method step 21 can be provided two-dimensionally or three-dimensionally for a plurality of instruments 2a and 2b, depending on the choice made for decision steps 18 and 19.

Following the image display in method step 21, the location method can be restarted according to decision step 22 to update the position and location of the medical instrument 2a or 2b. If this is not necessary, the method ends after the display of the position and location of the medical instrument 2a or 2b in method step 21.

Known methods, e.g. filter methods, can then also be used for further determining the varied position and location of the medical instrument 2a or 2b.

1-21. (canceled)

22. A method for locating a medical instrument within an object during a medical procedure, comprising:
inserting the medical instrument at least partly into the object;
acquiring a first image of the object comprising the medical instrument under a first acquisition condition;
providing a first image data set of the first image;
acquiring a second image of the object comprising the medical instrument under a second acquisition condition;
providing a second image data set of the second image; and
subtracting the second image data set from the first image data set.

23. The method as claimed in claim 22, wherein:
the first image is acquired without a contrast medium and the second image is acquired with the contrast medium, or
the first image is acquired with the contrast medium and the second image is acquired without the contrast medium.

24. The method as claimed in claim 23, wherein the contrast medium is injected into the medical instrument for producing a contrast enhancement:
in an area surrounding the medical instrument, or
inside the medical instrument.

25. The method as claimed in claim 22, wherein the first acquisition condition differs from the second acquisition condition by a position or a location of an instrument marking arranged on the medical instrument.

26. The method as claimed in claim 25, wherein the instrument marking is arranged on a collapsible, rotatable or alignable tip of the medical instrument.

27. The method as claimed in claim 22, wherein the first or second acquisition condition is initiated time-adjustably.

28. The method as claimed in claim 22, wherein the first and second images are acquired as a two-dimensional projection of the object and a two-dimensional position or location of the medical instrument is determined therefrom.

29. The method as claimed in claim 28, wherein a three-dimensional position or location of the medical instrument is identified from at least two different two-dimensional projections of the object.

30. The method as claimed in claim 29, wherein a plurality of views are identified for the three-dimensional position or location of the medical instrument.

31. The method as claimed in claim 22, wherein the first and second images are acquired as a three-dimensional data of the object and a three-dimensional position or location of the medical instrument is determined therefrom.

32. The method as claimed in claim 31, wherein a plurality of views are identified for the three-dimensional position or location of the medical instrument.

33. The method as claimed in claim 22, wherein a starting position or starting location of the medical instrument is determined by the image subtraction and a variation of the position or location of the medical instrument is determined by acquiring a further image of the object without the image subtraction.

34. The method as claimed in claim 22, wherein the images are acquired using a method selected from the group consisting of: a magnetic resonance method, an X-ray method, and an ultrasound method.

35. The method as claimed in claim 22, wherein a patient marking is captured on the first and second images.

36. The method as claimed in claim 22, wherein a starting position or starting location and a variation of a position or location of a plurality of medical instruments are determined sequentially.

37. The method as claimed in claim 22, wherein the first and second images are acquired with the object at an identical condition of motion.

38. The method as claimed in claim 22, wherein a background deviation of substantially identical images is corrected by an image-based elastic registration.

39. The method as claimed in claim 22, wherein the object is a human or animal patient.

40. A medical arrangement for locating a medical instrument within a patient during a medical procedure, comprising:
an injection system which injects a contrast medium into the medical instrument;
an image device which acquires a first and second images of the patient comprising the medical instrument under a first and second acquisition conditions;
a monitoring system which monitors a status of the patient;
an image data processing system which subtracts the second image from the first image; and
a display device which displays the first and second images.

41. The medical arrangement as claimed in claim 40, wherein:
the first image does not comprises the contrast medium and the second image comprises the contrast medium, or
the first image comprises the contrast medium and the second image does not comprises the contrast medium.
